(19) United States
(12) Patent Application Publication
(10) Pub. No.: US 2016/0030567 A1
(43) Pub. Date: Feb. 4, 2016

(54) Topical Homeopathic Remedies for Generalized Dermatitis

(71) Applicant: Carolyn Blakeslee, Anthony, FL (US)

(72) Inventor: Carolyn Blakeslee, Anthony, FL (US)

(21) Appl. No.: 14/450,686

(22) Filed: Aug. 4, 2014

(51) Int. Cl. A61K 47/02; A61K 33/10; A61K 35/24; A61K 36/38; A61K 36/83; A61K 36/22; A61K 36/28; A61K 35/763

(52) U.S. Cl. CPC A61K 47/02; A61K 36/28; A61K 33/10; A61K 35/763; A61K 36/38; A61K 36/83; A61K 36/22; A61K 35/24

(57) ABSTRACT

The present invention relates to a topical homeopathic remedy for skin conditions generally known as dermatitis. The present invention further relates to a composition forming said topical homeopathic remedy, a process for creating such composition and a method of using same. The present invention may comprise a combination of at least one of the following compounds: arnica montana, calcarea carbonica, calendula officinalis, herpes zoster, hypericum, mezereum, rhus toxicodendron, and variolinum, polymerized in a mixture of calcium montmorillonite and water.

### TECHNICAL FIELD

The present invention relates to a natural, topical homeopathic remedy for skin conditions generally known as dermatitis. The present invention further relates to a composition forming said topical homeopathic remedy, a process for creating such composition and a method of using same.

### BACKGROUND OF THE INVENTION

Natural skin care has its roots in the 4th millennium BC in Egypt with natural skin care treatments for a variety of skin conditions. One such treatment consists of bullock's bile, whipped ostrich eggs, olive oil, dough and resin mixed with milk. In the modern age many people with unique skin types and needs, for example, sensitive skin, dry skin, oily skin or a combination thereof, have turned to natural skin care solutions. Some examples of natural skin care ingredients include jojoba, safflower oil, rose hip seed oil, shea butter, beeswax, witch hazel, aloe vera, tea tree oil, and chamomile. Many of these natural ingredient combinations can be tailored specifically to the individual's skin type or skin condition. There is, however, no actual definition of “natural” according to the U.S. Food and Drug Administration (FDA). All ingredients are chemicals by definition. “Derived” ingredients are unnatural both according to the original substance and the method of derivation.

### SUMMARY OF THE INVENTION

It is, therefore, an object of the present invention to provide a composition for a topical homeopathic remedy for skin conditions generally known as dermatitis. It is another object of the present invention to provide a process for creating such composition and a method of using same.

A preferred embodiment of the present invention relates to a topical homeopathic remedy comprising a polymer.

In one exemplary aspect of the preferred embodiment, the polymer further comprises a combination of at least one of the following compounds: arnica montana, calcarea carbonica, calendula officinalis, herpes zoster, hypericum, mezereum, rhus toxicodendron, and valriolinum, polymerized in a mixture of calcium montmorillonite and water.

The following are additional and/or exemplary aspects of the exemplary topical homeopathic remedy, as described above, one or more of which can be combined with the basic invention as embodied above:
the combination of the at least one compound is diluted by a factor of at least 10⁻⁶⁰ or thirty on a centesimal scale (30 C);
the combination of the at least one compound further comprises a combination of all said compound; and
the combination of the at least one compound is a colloid.

One exemplary method of creating a topical homeopathic remedy polymer can optionally comprise any, all, or one of the following steps, in any order of operation:
diluting at least one compound comprising arnica montana, calcarea carbonica, calendula officinalis, herpes zoster, hypericum, mezereum, rhus toxicodendron, and valriolinum in distilled water;
agitating the resultant diluted at least one compound with a mixture of calcium montmorillonite and distilled water;
further agitating said combination of the resultant diluted at least one compound the mixture of calcium montmorillonite until a desired combination is achieved.

The following are additional and/or exemplary aspects of the method, as described above, one or more of which can be combined with the basic invention as embodied above:
the combination of the at least one compound is diluted by a factor of at least 10⁻⁶⁰ or thirty on a centesimal scale (30 C);
the combination of the at least one compound further comprises a combination of all said compounds; and
the desired combination is a colloid.

One exemplary method of using a topical homeopathic remedy can comprising any, all, or none of the following steps of, in any order of operation:

cleaning an area of an epidermis, wherein cleaning comprises removing any debris, irritants, or other non-naturally occurring materials from the surface of the epidermis using an astringent, alcohol, water, or other epidermis cleaning agent;

extracting an amount of the polymer from a container;

applying the polymer directly to the area of the epidermis; and

reapplying the polymer on the area of the epidermis as needed.

The following are additional and/or exemplary aspects of the method, as described above, one or more of which can be combined with the basic invention as embodied above:
the polymer further comprising a combination of at least one of the following compounds: arnica montana, calcarea carbonica, calendula officinalis, herpes zoster, hypericum, mezereum, rhus toxicodendron, and valriolinum, polymerized in a mixture of calcium montmorillonite and water;
the combination of the at least one compound is diluted by a factor of at least 10⁻⁶⁰ or thirty on a centesimal scale (30C);
the combination of the at least one compound further comprises a combination of all said compounds; and
the combination of the at least one compound is a colloid.

These and other exemplary aspects of the present invention are described herein below. Those skilled in the art will recognize still other aspects of the present invention upon reading and understanding the attached description.

### DETAILED DESCRIPTION OF THE INVENTION

The present invention will now be described more fully herein after with reference to the examples below and any illustrations and/or tables contained therein, which form a part hereof, and which show, by way of specific exemplary embodiments by which the invention may be practiced. This invention may, however, be embodied in many different forms and should not be construed as limited to the exemplary embodiments set forth herein. Rather, these exemplary embodiments are provided so that this disclosure will be thorough and complete, and will fully convey the scope of the invention to those skilled in the art. The following detailed description, is, therefore, not to be taken in a limiting sense.

Throughout the specification and claims, the following terms take the meanings explicitly associated herein, unless the context clearly otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

The following briefly describes the embodiments of the invention in order to provide a basic understanding of some aspects of the invention. This brief description is not intended as an extensive overview. It is not intended to identify key or critical elements, or to delineate or otherwise narrow the scope, its purpose is merely to present some concepts in a simplified form as a prelude to the more detailed description that is presented later.

The present invention, generally, is directed towards a topical homeopathic epidermis treatment for general and common human skin conditions, generally known as dermatitis. The present invention is a composition of a number of natural chemical compounds which, when combined together with one another, form a novel and useful skin care treatment product. As described in more detail below, the combination of chemical compounds may occur in varying formulas and amounts, and all compounds are not necessary and interchangeable with one another, so as to create novel and individual compositions. For example, all compounds as described below may be contained in said composition, none of said compounds, or any combination of said compounds may be used.

Dermatitis is generally defined as inflammation of the skin. It is characterized by itchy, erythematous, vesicular, weeping, and crusting patches. The term may also be synonymous with the condition eczema, or what is also commonly used to describe atopic dermatitis or atopic eczema. Like previously stated, dermatitis and eczema may be considered synonymous conditions, or may also be defined, for example, where dermatitis implies an acute condition and eczema a chronic one.

The terms are broadly applied to a range of persistent skin conditions. These include dryness and recurring skin rashes that are characterized by one or more of these symptoms: redness, skin swelling, itching and dryness, crusting, flaking, blistering, cracking, oozing, or bleeding. Areas of temporary skin discoloration may appear and are sometimes due to healed injuries. Scratching open a healing lesion may result in scarring and may enlarge the rash.

Common examples of dermatitis include atopic dermatitis (aka infantile e., flexural e., atopic dermatitis) is an allergic disease believed to have a hereditary component, and often runs in families whose members also have asthma. Itchy rash is particularly noticeable on head and scalp, neck, inside of elbows, behind knees, and buttocks. It is very common in developed countries, and rising.

Contact dermatitis is of two types: allergic (resulting from a delayed reaction to an allergen, such as poison ivy, nickel, or Balsam of Peru), and irritant (resulting from direct reaction to a detergent, such as sodium lauryl sulfate, for example). Some substances act both as allergen and irritant (wet cement, for example). Other substances cause a problem after sunlight exposure, bringing on phototoxic dermatitis. About three quarters of cases of contact eczema are of the irritant type, which is the most common occupational skin disease. Contact eczema is curable, provided the offending substance can be avoided and its traces removed from one's environment.

Xerotic eczema (aka asteatotic e., e. craquele or craquelatum, winter itch, pruritus hiemalis) is dry skin that becomes so serious it turns into eczema. It worsens in dry winter weather, and limbs and trunk are most often affected. The itchy, tender skin resembles a dry, cracked, river bed. This disorder is very common among the older population. Ichthyosis is a related disorder.

Seborrhoeic dermatitis or Seborrheic dermatitis (“cradle cap” in infants) is a condition sometimes classified as a form of eczema that is closely related to dandruff. It causes dry or greasy peeling of the scalp, eyebrows, and face, and sometimes trunk. The condition is harmless except in severe cases of cradle cap. In newborns it causes a thick, yellow, crusty scalp rash called cradle cap, which seems related to lack of biotin and is often curable.

The present invention, as described by examples in more detail below, contains varying combinations of the following naturally occurring compounds to aid with relieving pain, discomfort, discoloration and various side effects of dermatitis variations as described above. Said compounds, which have the commonly ascribed names stated below, are as follows: calcium montmorillonite; water; arnica Montana, calcarea carbonica, calendula officinalis, herpes zoster, hypericum perforatum, mezereum, rhus toxicondendron, and variolimum.

Montmorillonite ((Na,Ca)_{0.33} (Al,Mg)₂(Si₄O₁₀)(OH)₂.nH₂O) is a very soft phyllosilicate group of minerals that typically form in microscopic crystals, forming a clay. Montmorillonite generally has color properties that are, for example, White, pale pink, blue, yellow, red, green. Montmorillonite is a member of the smectite group, and is commonly known as a 2:1 clay, meaning that it has 2 tetrahedral sheets sandwiching a central octahedral sheet. The particles are plate-shaped with an average diameter of approximately one micrometre. Members of this group may include, for example, saponite.

Montmorillonite is a subclass of smectite, a 2:1 phyllosilicate mineral being characterized as having greater than 50% octahedral charge; its cation exchange capacity is due to isomorphous substitution of Mg for Al in the gibbsitic plane. In contrast, beidellite is smectite with greater than 50% tetrahedral charge originating from isomorphous substitution of Al for Si in the quartz sheet.

The water content of montmorillonite is variable and it increases greatly in volume when it absorbs water. Chemically it is hydrated sodium calcium aluminium magnesium silicate hydroxide (Na,Ca)_{0.33}(Al,Mg)₂(Si₄O₁₀)(OH)₂.nH₂O. Potassium, iron, and other cations are, for example, common substitutes, the exact ratio of cations varies with source. It often occurs intermixed with chlorite, muscovite, illite, cookeite, and kaolinite.

Water is a transparent fluid which forms the world's streams, lakes, oceans and rain, and is the major constituent of the fluids of living things. As a chemical compound, a water molecule contains one oxygen and two hydrogen atoms that are connected by covalent bonds. Water is a liquid at standard ambient temperature and pressure, but it also often commonly co-exists in a solid state known as ice and and a gaseous state, known as steam or water vapor. It is contemplated for use as part of this invention in its liquid state. Furthermore, it is contemplated to have a composition known as distilled water, filtered water, or any other cleansing and/or removal of impurities which are not beneficial to the overall quality of the present invention yet is still capable of being created and is functionally necessary as a component of the present invention.

Arnica montana, known commonly as leopard's bane, wolf s bane, mountain tobacco and mountain arnica, is a European flowering plant with large yellow capitula. This plant has tall stems, 20-60 centimeters high, supporting, commonly, a single flower head. The leaves are generally in a basal rosette, but one or two pairs may be found on the stem and are, unusually for composites, opposite. The flower heads are yellow, approximately 5 centimeters in diameter. Arnica montana has been in use since the 1500s, according to the University of Maryland Medical Center. Creams, ointments and gels containing homeopathic arnica are intended for topical use. Arnica is best known for its effect on bruising and swelling. Arnica montana may be, for example, ingested in a regulated quantity for an injury or fall to minimize the development of edema and swelling. Arnica may also be applied to directly to the skin for pain and swelling associated with bruises, aches, sprains, and arthritis. Additional uses are application to the skin for insect bites, muscle and cartilage pain, chapped lips, and acne.

Calcarea carbonica, also generally known as calcium carbonate is a chemical compound with the formula CaCO₃. It is a common substance found in rocks in all parts of the world, and is the main component of shells of marine organisms, snails, coal balls, pearls, and eggshells. Calcium carbonate is the active ingredient in agricultural lime, and is created when Ca ions in hard water react with carbonate ions creating limescale. It is commonly used medicinally as a calcium supplement or as an antacid, but excessive consumption can be hazardous.

Calcium carbonate is widely used medicinally as an inexpensive dietary calcium supplement or gastric antacid. It may be used as aphosphate binder for the treatment of hyperphosphatemia (primarily in patients with chronic renal failure). It is also used in the pharmaceutical industry as an inert filler for tablets and other pharmaceuticals.

Calcium carbonate is used in the production of toothpaste and has seen a resurgence as a food preservative and color retainer, when used in or with products such as organic apples or food. Excess calcium from supplements, fortified food and high-calcium diets, can cause the milk-alkali syndrome, which has serious toxicity and can be fatal. Calcium has been added to over-the-counter products, which contributes to inadvertent excessive intake.

Calendula officianalis is a short-lived aromatic herbaceous perennial, growing to approximately 80 centimeters (31 inches) tall, with sparsely branched lax or erect stems. The leaves are oblong-lanceolate, hairy on both sides, and with margins entire or occasionally waved or weakly toothed. The inflorescences are yellow, comprising a thick capitulum or flowerhead which commonly has a 4-7 centimeter diameter surrounded by two rows of hairy bracts; in the wild plant they have a single ring of ray florets surrounding the central disc florets. The disc florets are tubular and hermaphrodite, and generally of a more intense orange-yellow colour than the female, tridentate, peripheral ray florets. The flowers may appear all year long where conditions are suitable. The fruit is a thorny curved achene. Pot marigold florets are edible. They are often used to add color to salads or added to dishes as a garnish and in lieu of saffron. The leaves are edible but are often not palatable. They have a history of use as a potherb and in salads. Calendula extracts may have anti-viral, anti-genotoxic, and anti-inflammatory properties in vitro. In an in vitro assay, the methanol extract of C. officinalis exhibited antibacterial activity and both the methanol and the ethanol extracts showed antifungal activities. Along with horsetails (Equisetum arvense), pot marigold is one of the few plants which is considered astringent despite not being high in tannins.

Herpes zoster (or simply zoster), commonly known as shingles and also known as zona, is a viral disease characterized by a painful skin rash with blisters in a limited area on one side of the body (left or right), often in a stripe. The initial infection with varicella zoster virus (VZV) causes the acute, short-lived illness chickenpox which generally occurs in children and young adults. Once an episode of chickenpox has resolved, the virus is not eliminated from the body and can go on to cause herpes zoster often many years after the initial infection. Herpes zoster is not the same disease as herpes simplex, despite the name similarity; both the varicella zoster virus and herpes simplex virus belong to the same viral subfamily Alphaherpesvirinae. Elements and derivatives of this bacterial strain can be separated and combined with other compounds to increase the transmittability and absorption of other compounds.

Hypericum performatum is a yellow flowering, stoloniferous or sarmentose, perennial herb The herb's common name comes from its traditional flowering and harvesting on St. John's day, 24 June. The genus name Hypericum is derived from reference to the plant's traditional use in warding off evil by hanging plants over a religious icon in the house during St. John's day. The species name perforatum refers to the presence of small oil glands in the leaves that look like windows, which can be seen when they are held against the light. St. John's wort is a perennial plant with extensive, creeping rhizomes. Its stems are erect, branched in the upper section, and can grow to 1 m high. It has opposing, stalkless, narrow, oblong leaves that are 12 millimeters long or slightly larger. The leaves are yellow-green in color, with transparent dots throughout the tissue and occasionally with a few black dots on the lower surface. Leaves exhibit obvious translucent dots when held up to the light, giving them a ‘perforated’ appearance. The flowers measure up to 2.5 centimeters across, commonly have five petals, and are colored bright yellow with conspicuous black dots. The flowers appear in broad cymes at the ends of the upper branches, between late spring and early to mid summer. The sepals are pointed, with glandular dots in the tissue. There are many stamens, which are united at the base into three bundles. The pollen grains are ellipsoidal. When flower buds (not the flowers themselves) or seed pods are crushed, a reddish/purple liquid is produced extracts of the plant are commonly sold over-the-counter as a treatment for depression.

Daphne mezereum, commonly known as mezereon, is a species of Daphne in the flowering plant family Thymelaeaceae. It is generally confined to soils derived from limestone. It is a deciduous shrub growing approximately up to 1.5 meters tall. The leaves are soft, generally 3-8 centimeters long and 1-2 centimeters broad, arranged spirally on the stems. The flowers are produced in early spring on the bare stems before the leaves appear. They have a four-lobed pink or light purple (rarely white) perianth, generally 10-15 millimeters in diameter, and are strongly scented. The fruit is a bright red berry commonly 7-12 millimeters in diameter; it is very poisonous for people. Mezereum is useful as a topical skin remedy, for dermatitis, inflammation, itching, crawling feeling, bedtime itching, and where irritation results on an area of skin following scratching. Mezereum is useful for nerve pain including the face, teeth and bone, as well as for skin ulcers with pus and vesicleas around the ulcer. Additional uses for Mezereum are for liver spots, to resolve rashes, red rashes with violent itching, to reduce bruising and helps with sinus pressure and pain.

Rhus toxicodendron Rhus toxicodendron can also be used as a homeopathic remedy. It is also known mostly generally by its more common name, poison ivy. Other names include Toxicodendron pubescens and Toxicodendron radicans. Rhus toxicodendron is used for treating several conditions, including: skin rashes, cramps, strains, sprains, restless leg syndrome, flu, viral infections, and arthritis.

Variolinum is the contents of the ripened pustule of smallpox, which may be reasonably prepared so as to be homeopathically beneficial.

### ONE PREFERRED EMBODIMENT

TABLE 1

| Compound | Amount |
|---|---|
| Arnica Montana | 30C |
| Calcarea Carbonica | 30C |
| Calendula Officinalis | 30C |
| Herpes Zoster (nosode) | 30C |
| Hypericum Perforatum | 30C |
| Mezereum | 30C |
| Rhus Toxicodendron | 30C |
| Variolinum (nosode) | 30C |

Homeopathy involves a process known by practitioners as dynamisation or potentisation, whereby a substance can be diluted with a liquid, such as for example, alcohol, distilled water or other liquid. The dilution occurs by a process of vigorously shaking the substance in the liquid, a process called succussion. Several potency scales are in use in homeopathy. For the preferred embodiment, the centesimal or “C scale” is used by way of example, whereby, each compound is created by diluting a substance by a factor of 100 at each stage. By way of illustration, a 2C dilution requires a substance to be diluted to one part in one hundred, and then a portion of that diluted solution diluted by a further factor of one hundred. A resulting suspension is one part of the original substance in 10,000 parts of the solution. In another similar illustration, a 6C dilution repeats this process six times, ending up with the original material diluted by a factor of 100⁻⁶=10⁻¹².

Furthermore, higher dilutions follow the same pattern. In homeopathy, a solution that is more dilute is described as having a higher potency, and more dilute substances are considered by homeopathic practitioners to be stronger and deeper-acting remedies. 30C is the measurement shown in Table 1, as 30C is commonly considered to be an optimal dilution for homeopathic preparations. Additional volumes and/or measurements are further considered, as described below, and 30C is used here, generally, only in illustration.

Any combination of the compounds, also known as polymers, listed above can be combined with a solution of calcium montmorillonite and water using any viable combination method, including, but not limited to: emulsion, dispersion, polymerization, or any other functionally compatible combining method.

A polymer is commonly and generally defined as a large molecule, or macromolecule, composed of many repeated sub-units. The term polymer covers a number of materials and compositions because of the broad range of properties that may occur within articles under this broad definition. Both synthetic and natural polymers play an essential and ubiquitous role in everyday life. Polymers range from familiar synthetic plastics, for example, such as polystyrene to natural biopolymers, for example, such as DNA and proteins that are fundamental to biological structure and function. The term polymer also refers to a molecule whose structure is composed of multiple repeating units, from which originates a characteristic of high relative molecular mass and attendant properties. The units composing polymers derive, actually or conceptually, from molecules of low relative molecular mass.

Historically, products arising from the linkage of repeating units by covalent chemical bonds have been the primary focus of polymer science. In biological contexts, essentially all biological macromolecules, for example, proteins (polyamides), nucleic acids (polynucleotides), and polysaccharides—are purely polymeric, or are composed in large part of polymeric components—e.g., isoprenylated/lipid-modified glycoproteins, where small lipidic molecule and oligosaccharide modifications occur on the polyamide backbone of the protein.

An emulsion dispersion comprises polymers, thermoplastics, and/or elastomers suspended in a waterphase with help of emulsifiers. An emulsion is a mixture of two or more liquids that are normally immiscible (i.e., nonmixable or unblendable). Emulsions are part of a more general class of two-phase systems of matter called colloids. Although the terms colloid and emulsion are sometimes used interchangeably, emulsion should be used when both the dispersed and the continuous phase are liquids. In an emulsion, one liquid (the dispersed phase) is dispersed in the other (the continuous phase). Non-limiting examples of emulsions include but are not limited to, vinaigrettes, milk, mayonnaise, and some cutting fluids for metal working. The word “emulsion” comes from the Latin word for “to milk”, as milk is an emulsion of milk fat and water, among other components.

Furthermore, two or more liquids can form different types of emulsions. As an example, oil and water can form, first, an oil-in-water emulsion, wherein the oil is the dispersed phase, and water is the dispersion medium. Second, they can form a water-in-oil emulsion, wherein water is the dispersed phase and oil is the external phase.

Emulsions, being liquids, do not exhibit a static internal structure. The droplets dispersed in the liquid matrix (called the “dispersion medium”) are usually assumed to be statistically distributed.

Where any of the example compounds given in Table 1 are in liquid form, combination of the compounds may result in an intermediate emulsion which is further combinable or usable separate and/or independent from said created polymer of combination(s) created thereof.

Dispersion is a less invasive or artificial means of distribution a compound throughout calcium montmorillonite. Dispersion refers to the distribution of a particular chemical constituent or element in a particular substance, where the concentration of the element gradually reaches the average value, also commonly known as a background value, from high concentrations with respect to space. Dispersion can be considered as primary or secondary, based on formational environment. Dispersion commonly occurs where a substance is sodic, for example calcium montmorillonite is sodic, the particles of the montmorillonite are forced apart. This is generally a major cause of erosion and breakdown of the montmorillonite composition if dispersion does not occur in controlled conditions.

Polymers, both natural and synthetic as defined above, are both created and combined via polymerization of many small molecules, known as monomers. Their consequently large molecular mass relative to small molecule compounds produces unique physical properties, including toughness, viscoelasticity, and a tendency to form glasses and semicrystalline structures rather than crystals. In chemical compounds, for example, such as arnica Montana, calcarea carbonica, calendula officinalis, herpes zoster, hypericum perforatum, mezereum, rhus toxicondendron, and variolinum in any varying dilution, polymerization occurs via a variety of reaction mechanisms that vary in complexity due to functional groups present in reacting compounds and their inherent steric effects. In more a traditional, straightforward polymerization, in one example, alkenes, which are relatively stable due to σ bonding between carbon atoms, form polymers through relatively simple radical reactions; in contrast, in a more complex reaction example, such as those that involve substitution at the carbonyl group require more complex synthesis due to the way in which reacting molecules polymerize.

As alkenes can be formed in somewhat straightforward reaction mechanisms, they form useful compounds such as polyethylene and polyvinyl chloride (PVC) when undergoing radical reactions, which are produced in high tonnages each year due to their usefulness in manufacturing processes of commercial products, such as piping, insulation and packaging. Other monomer units, such as formaldehyde hydrates or simple aldehydes, are able to polymerize themselves at quite low temperatures (ca. −80° C.) to form trimers, or molecules consisting of 3 monomer units, which can cyclize to form ring cyclic structures, or undergo further reactions to form tetramers, or 4 monomer-unit compounds. Further compounds either being referred to as oligomers in smaller molecules.

Polymerization that is not sufficiently moderated and proceeds at a fast rate can be very hazardous. This phenomenon is known as hazardous polymerization and can cause fires and explosions. Therefore, the combination of said compound polymers listed in Table 1, should be combined at a molecular level in a manner and fashion which adequately combines said compounds in such a manner as to retain all beneficial qualities to said compounds independently, but still activate the necessary reactions which create the novelty of this present invention—without limiting the combining method to one such method, but to any and all functionally necessary combination methods which are compatible with said polymer compounds.

In this preferred embodiment, the resulting combination of compounds as listed in Table 1 should result in a colloid mixture. A colloid is a substance in which microscopically dispersed insoluble particles are suspended throughout another substance. Sometimes the dispersed substance alone is called the colloid, the term colloidal suspension refers unambiguously to the overall mixture. A narrower definition of the word suspension, as defined below, is contradistinguished from colloids by larger particle size. Unlike a solution, whose solute and solvent constitute only one phase, a colloid has a dispersed phase (the suspended particles) and a continuous phase (the medium of suspension). To qualify as a colloid, the mixture must be one that does not settle or would take a very long time to settle appreciably. In this preferred embodiment, the medium of suspension or the continuous phase would be the calcium montmorillonite clay; whereas the suspended particles or the dispersed phase would be the agitated and/or otherwise combined arnica Montana, calcarea carbonica, calendula officinalis, herpes zoster, hypericum performatum, mezereum, rhus toxicondendron and/or variolinum.

The dispersed phase particles may have, for example, a diameter of between approximately 1 and 1000 nanometers. The particle diameter may also, for example be dependent on the potency of dilution, as discussed above. Such particles are normally easily visible in an optical microscope, although at the smaller size range (r<250 nm), an ultramicroscope or an electron microscope may be required. Homogeneous mixtures with a dispersed phase in this size range may be called colloidal aerosols, colloidal emulsions, colloidal foams, colloidal dispersions, or hydrosols. The dispersed-phase particles or droplets are affected largely by the surface chemistry present in the colloid. Some colloids are translucent because of the Tyndall effect, which is the scattering of light by particles in the colloid. Other colloids may be opaque or have a slight color.

In additional embodiments, the combination of the compounds listed in Table 1 can also result in a chemical state known as a suspension where varying dilutions of said compound polymers are used. A suspension is a heterogeneous mixture containing solid particles that are sufficiently large for sedimentation. Usually they must be larger than 1 micrometer. For example, sedimentation may occur where the calcium montmorillonite clay is initially in a powder form, and the other compounds are in a liquid form. The internal phase (i.e., the solid calcium montmorillonite) is dispersed throughout the external phase (i.e., the remaining fluid elements) through, for example, mechanical agitation. Also, excipients or suspending agents may be necessary additives to said polymer reaction in embodiments with varying dilution of compound elements. Unlike colloids, suspensions will eventually settle. An example of a suspension would be sand in water. The suspended particles are visible under a microscope and will settle over time if left undisturbed. This distinguishes a suspension from a colloid, in which the suspended particles are smaller and do not settle. Furthermore, colloids and suspensions are different from solutions, in which the dissolved substance (solute) does not exist as a solid, and solvent and solute are homogeneously mixed.

Suspensions are unstable from the thermodynamic point of view; however, they can be kinetically stable over a large period of time, which determines their shelf life. This time span needs to be measured to ensure the best product quality to the final consumer.

In the preferred embodiment, one exemplary method of creating a the polymer, comprises a first step of diluting at least one of the compounds as described in Table 1 to create the necessary dilutions. As discussed prior, dilution ratio and amount of each compound may vary depending on a number of variables as inherent to the mixing of the compounds. A second step may include agitating the resultant compound dilution(s) with a mixture of calcium montmorillonite and distilled water. Additionally, further agitating the resultant mixture should occur until a desired combination is achieved.

Lastly, a method of using a topical homeopathic remedy is further contemplated, including cleaning an area of an epidermis. Cleaning may include, but is not limited to, removing any debris, irritants, or other non-naturally occurring materials from the surface of the epidermis using an astringent, alcohol, water, or other epidermis cleaning agent. Thereafter, an amount of the remedy can be extracted from a container holding the remedy. It is contemplated that the container is air-tight and impermeable, so as to limit the amount of natural deterioration of the remedy as is functionally necessary to maintain shelf-life. The polymer can then be applied directly to the area of the epidermis, and reapplication as necessary.

It will be apparent to one of ordinary skill in the art that the manner of making and using the claimed invention has been adequately disclosed in the above-written description of the exemplary preferred embodiment and other aspects. It should be understood, however, that the invention is not necessarily limited to the specific embodiment, described aspects, arrangement and components as described above, but may be susceptible to numerous variations within the scope of the invention.

Moreover, particular exemplary features described herein in conjunction with the preferred embodiment and/or aspects of the present invention are to be construed as applicable to any embodiment described within, enabled thereby, or apparent wherefrom. Thus, the specification is to be regarded in a broad, illustrative, and enabling sense, rather than a restrictive one.

Further, it will be understood that the above description of the embodiments of the present invention are susceptible to various modifications, changes, and adaptations, and the same are intended to be comprehended within the meaning and range of equivalents of the appended claims.

What I claim is:

1. A topical homeopathic remedy comprising a polymer, said polymer further comprising a combination of at least one of the following compounds: arnica montana, calcarea carbonica, calendula officinalis, herpes zoster, hypericum, mezereum, rhus toxicodendron, and variolinum, polymerized in a mixture of calcium montmorillonite and water.

2. The topical homeopathic remedy of claim 1, wherein the combination of the at least one compound is diluted by a factor of at least 10⁻⁶⁰ or thirty on a centesimal scale (30C).

3. The topical homeopathic remedy of claim 1, wherein the combination of the at least one compound further comprises a combination of all said compounds.

4. The topical homeopathic remedy of claim 1, wherein the combination of the at least one compound is a colloid.

5. A method of creating a topical homeopathic remedy polymer, comprising the steps of:
diluting at least one compound comprising arnica montana, calcarea carbonica, calendula officinalis, herpes zoster, hypericum, mezereum, rhus toxicodendron, and variolinum in water;
agitating the resultant diluted at least one compound with a mixture of calcium montmorillonite and water; and
further agitating said combination of the resultant diluted at least one compound the mixture of calcium montmorillonite until a desired combination is achieved.

6. The method of claim 5, wherein the combination of the at least one compound is diluted by a factor of at least 10⁻⁶⁰ or thirty on a centesimal scale (30C).

7. The method of claim 5, wherein the combination of the at least one compound further comprises a combination of all said compounds.

8. The method of claim 5, wherein the desired combination is a colloid.

9. A method of using a topical homeopathic remedy comprising a polymer, comprising the steps of:
cleaning an area of an epidermis, wherein cleaning comprises removing any debris, irritants, or other non-naturally occurring materials from the surface of the epidermis using an astringent, alcohol, water, or other epidermis cleaning agent;
extracting an amount of the polymer from a container;
applying the polymer directly to the area of the epidermis; and
reapplying the polymer on the area of the epidermis as needed.

10. The method of claim 9, wherein the polymer further comprising a combination of at least one of the following compounds: arnica montana, calcarea carbonica, calendula officinalis, herpes zoster, hypericum, mezereum, rhus toxicodendron, and variolinum, polymerized in a mixture of calcium montmorillonite and water.

11. The method of claim 10 , wherein the combination of the at least one compound is diluted by a factor of at least 10⁻⁶⁰ or thirty on a centesimal scale (30C).

12. The method of claim 10, wherein the combination of the at least one compound further comprises a combination of all said compounds.

13. The method of claim 10, wherein the combination of the at least one compound is a colloid.
